(19) United States
(12) Patent Application Publication
(10) Pub. No.: US 2026/0224160 A1
(43) Pub. Date: Aug. 6, 2026

(54) DEVICE AND METHOD IMPLEMENTED BY COMPUTER FOR THE MEASUREMENT OF ENDOUTERINE PARAMETERS

(71) Applicant: PROXENIA S.R.L., MODENA (IT)

(72) Inventor: Antonio LA MARCA, MODENA (IT)

(21) Appl. No.: 19/149,176

(22) PCT Filed: Jan. 22, 2024
(86) PCT No.: PCT/IB2024/050597

§ 371 Date: Jul. 18, 2025

(30) Foreign Application Priority Data
Jan. 20, 2023 (IT) 102023000000828

(51) Int. Cl. A61B 5/00

(52) U.S. Cl. CPC A61B 5/4325; A61B 5/0075; A61B 5/0086; A61B 5/6875; A61B 5/7239; A61B 5/7271

(57) ABSTRACT

The device (1) for the measurement of endouterine parameters, comprises:—at least one probe (2) insertable at least partly within a patient's uterus and comprising emitting means (2a) adapted to emit electromagnetic radiation to the uterus and receiving means (2b) adapted to receive reflected and/or scattered electromagnetic radiation from the uterus; and—optical detecting means (3) connected to the probe (2) and adapted to process the reflected and/or scattered electromagnetic radiation and to generate an electromagnetic spectrum representative of chemical and physical parameters of the uterus.

### TECHNICAL FIELD

The present invention relates to a device and a method implemented by computer for the measurement of endouterine biophysical and chemical parameters.

### BACKGROUND ART

It is known that suboptimal chemical and physical factors in a patient's uterus can impair the development of gametes and embryos, leading to a deep impact on their future development (Wale PL et al., Hum Reprod Update, 2016, 22:2-22).

The exact composition, variation and interdependent relationships of dissolved oxygen, dissolved carbon dioxide, pH and temperature within the female reproductive tract have to date been only partly investigated. Thus, the relevant concentrations of the various components and pH in commercially available culture media but also the concentration of oxygen, carbon dioxide and temperature currently used in incubators are very often based on extrapolated data mainly obtained from animal studies.

High, non-physiological oxygen concentrations can have a negative impact as far as oxidative stress is concerned, contributing to defective embryo development with higher fragmentation rates.

At the same time, too low levels of oxygen can impair embryogenesis, a process that involves many oxidative metabolic processes.

Other biophysical parameters, which could interact with the levels of dissolved oxygen and/or carbon dioxide, are also of primary importance in the embryo culture environment. pH is an important parameter for sperm binding and motility, oocyte maturation and embryo development. In vitro, biological pH buffers can be used to introduce into embryo culture media to help stabilize pH and minimize deleterious intracellular changes resulting from pH fluctuations in human pre-implantation embryos.

However, the in vivo regulation of pH in luminal fluids within the reproductive tract is more complex. From a clinical point of view, in humans, conditions of abnormal acidity or alkalinity within the reproductive tract are known to have some consequences on reproduction, although attempts to address this issue have been of limited success so far.

As to temperature, it is recognized that the extreme variation in body temperature causes an immune response, obtained through molecular networks of cytokines and microRNAs.

While embryos can demonstrate some degree of resilience to small changes in temperature, prolonged exposure in culture to temperatures other than the optimal 37 degrees can reduce rates of fertilization, implantation and completed pregnancies.

Given, therefore, the relevant role of the aforementioned chemical and physical parameters in the endouterine environment for the purpose of achieving efficient human reproduction, the need is felt to develop an appropriate technology which can enable the in vivo measurement of these parameters.

### DESCRIPTION OF THE INVENTION

The main aim of the present invention is to devise a device for the measurement of endouterine parameters which allows effective and accurate detection of chemical and physical parameters inside a uterus.

Another object of the present invention is to devise a device for the measurement of endouterine parameters which allows practical, in vivo detection of endouterine chemical and physical parameters.

A further object of the present invention is to devise a device for the measurement of endouterine parameters which allows rapid measurement and is well tolerated by the patient during use.

Still one object of the present invention is to devise a method implemented by computer for the measurement of endouterine parameters which is reliable and accurate.

Another object of the present invention is to devise a device and a method implemented by computer for the measurement of endouterine parameters which allow the aforementioned drawbacks of the prior art to be overcome within the framework of a simple, rational, easy and effective to use as well as affordable solution.

The aforementioned objects are achieved by this device for the measurement of endouterine parameters having the characteristics of claim 1.

The aforementioned objects are further achieved by the present method implemented by computer for the measurement of endouterine parameters having the characteristics of claim Y.

### BRIEF DESCRIPTION OF THE DRAWINGS

Other characteristics and advantages of the present invention will become more apparent from the description of a preferred, but not exclusive, embodiment of a device and method implemented by computer for the measurement of endouterine parameters, illustrated by way of an indicative, yet non-limiting example, in the accompanying tables of drawings in which:

FIG. 1 is a schematic representation of a device for the measurement of endouterine parameters according to the invention;

FIG. 2 is a schematic representation of a device for the measurement of endouterine parameters according to the invention;

FIG. 3 is a schematic representation of the probe insertion portion according to the invention;

FIG. 4 is a schematic detailed representation of the insertion portion according to the invention, in accordance with a first embodiment;

FIG. 5 is a schematic detailed representation of the insertion portion according to the invention, in accordance with a second embodiment;

FIG. 6 is a schematic detailed representation of the insertion portion according to the invention, in accordance with a third embodiment.

### EMBODIMENTS OF THE INVENTION

With particular reference to these figures, reference numeral 1 globally denotes a device for the measurement of endouterine parameters.

The device 1 according to the invention comprises at least one probe 2 insertable at least partly within a patient's uterus.

The probe 2 comprises emitting means 2a adapted to emit electromagnetic radiation to the uterus and receiving means 2b adapted to receive reflected and/or scattered electromagnetic radiation from the uterus.

In detail, electromagnetic radiation comprises infrared radiation, visible light radiation and/or ultraviolet radiation.

The device 1 also comprises optical detecting means 3 connected to the probe 2 and adapted to process the reflected and/or scattered electromagnetic radiation and to generate an electromagnetic spectrum representative of chemical and physical parameters of the uterus.

Specifically, the optical information (fluorescence decay time, optical intensity, spectrum, polarization, etc.) that can be derived from the electromagnetic spectrum obtainable by means of this device 1 allows determining the endometrial mucosal conditions. The aforementioned optical information is derived from specific conditions of the endometrial mucosa which may vary depending on a plurality of chemical and physical parameters such as pH, temperature, oxygenation, etc.

In accordance with a preferred embodiment, the optical detecting means 3 comprise infrared detection means configured to generate an infrared absorption spectrum.

Conveniently, the emitting means 2a comprise at least one emitting optical fiber 4 connected to a radiation source 5 and adapted to emit the electromagnetic radiation.

Specifically, the device 1 also comprises the radiation source 5 configured to emit the electromagnetic radiation which is transmitted to the uterus by means of the emitting optical fiber 4.

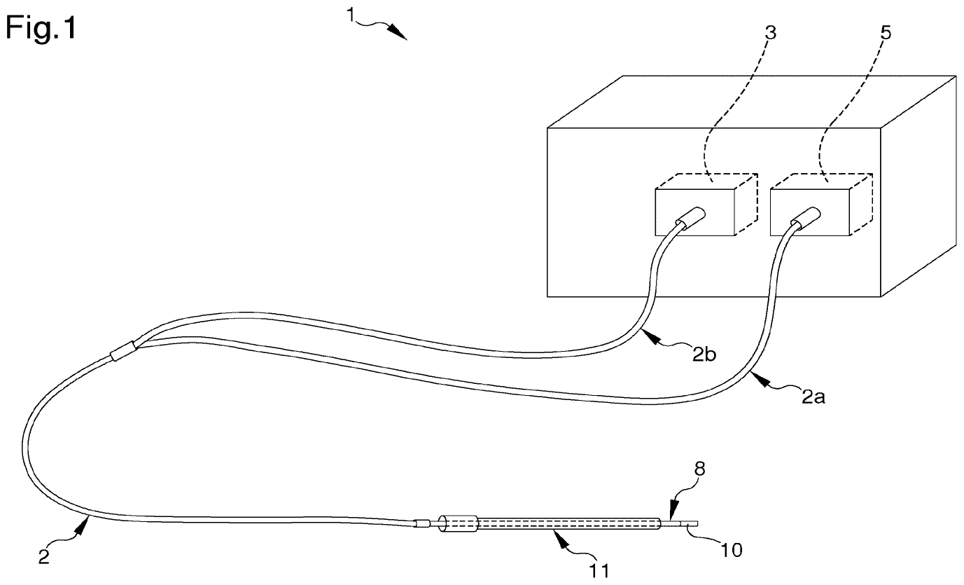

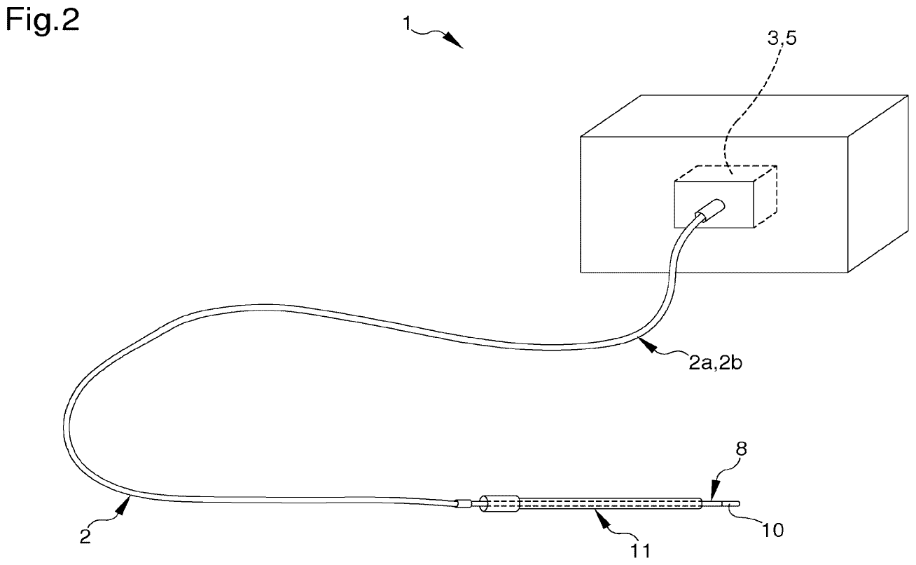

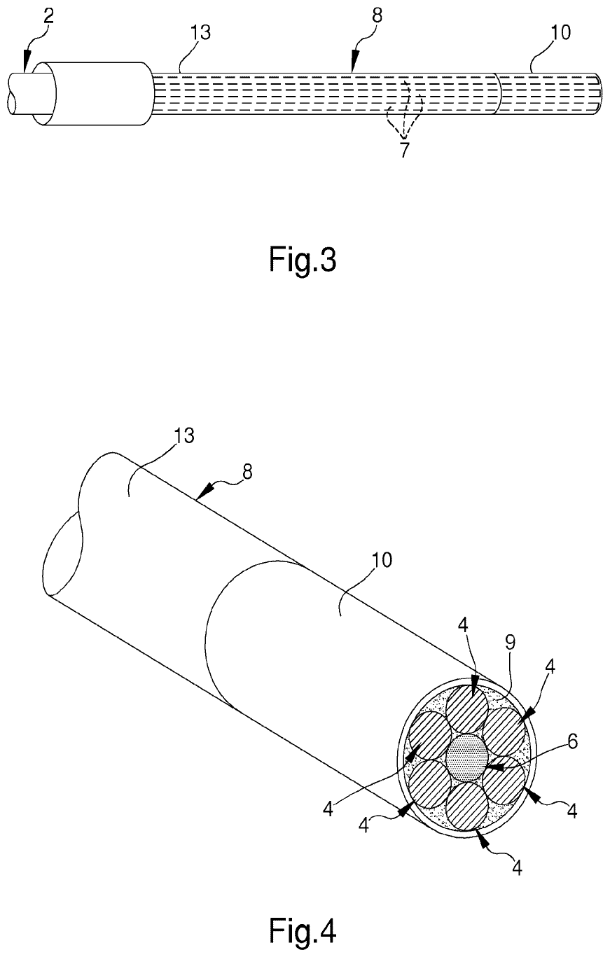

In accordance with the embodiment shown in FIG. 1, the radiation source 5 and the optical detecting means 3 are made in the form of separate devices. In accordance with an alternative embodiment shown in FIG. 2, however, the radiation source 5 and the optical detecting means 3 are made in the form of an individual device.

In accordance with the preferred embodiment, the radiation source 5 is configured to emit electromagnetic radiation having wavelengths belonging at least to the infrared spectrum.

The receiving means 2b comprise at least one receiving optical fiber 6 adapted to receive the reflected and/or scattered electromagnetic radiation and to transmit it to the optical detecting means 3.

The optical detecting means 3 comprise at least one optical detector such as e.g. photodiode, photomultiplier, spectrometer, spectrophotometer, charge-coupled device (CCD), active pixel sensor (CMOS).

The optical detecting means 3 also comprise at least one optical detecting fiber positioned between the receiving optical fiber 6 and the optical detector.

The optical detecting means 3 are configured to filter the reflected and/or scattered electromagnetic radiation and to process radiation having wavelengths of interest.

The optical detecting means 3 will be described in more detail later on in this disclosure.

Each of the optical fibers 4,6 comprises at least one central core configured to transmit electromagnetic radiation having a wavelength of between 300 nm and 2200 nm, and an outer casing to the central core. In detail, the central core has a refractive index so as to allow the electromagnetic radiation having a wavelength of between 300 nm and 2200 nm to be transmitted.

Conveniently, the emitting optical fiber 4 has a section wherein the total diameter, defined by the central core and by the casing, is of between 100 μm and 1 mm, wherein the diameter of the central core is of between 30 μm and 900 μm. Preferably, the total diameter, defined by the central core and by the casing, is 220 μm and the diameter of the central core is 200 μm.

The emitting optical fiber 4 has a coating made of plastic material. Preferably, the emitting optical fiber 4 has a coating made of polyimide.

The receiving optical fiber 6 has a section wherein the total diameter, defined by the central core and by the casing, is of between 100 μm and 1 mm, wherein the diameter of the central core is of between 30 μm and 900 μm.

Specifically, the diameter of the receiving optical fiber 6 is selected depending on the diameter of the detecting optical fiber of the optical detecting means 3 and must be greater than or equal to the latter.

Advantageously, the receiving optical fiber 6 has a coating adapted to optically isolate the receiving fiber itself and to optimize the signal transmission.

The coating of the receiving optical fiber 6 is made of plastic or metal material. Preferably, the coating of the receiving optical fiber 6 is made of aluminum. Advantageously, the emitting means 2a comprise a plurality of emitting optical fibers 4.

In accordance with a first embodiment shown in FIG. 4, the emitting means 2a comprise between five and seven, in this case six, emitting optical fibers 4. The number of six emitting optical fibers 4 allows for optimal emission of the electromagnetic radiation and, at the same time, allows the diameter of the probe 2 to be reduced, thus limiting the possible discomfort experienced by the patient during use.

Likewise, it should be specified that, for this purpose, the diameter of the optical fibers 4,6 is also appropriately selected so as to limit the total diameter of the probe 2 as much as possible.

Advantageously, the emitting optical fibers 4 are arranged around the receiving optical fiber 6. In detail, the emitting optical fibers 4 are arranged along a circumference having the receiving optical fiber 6 as its center.

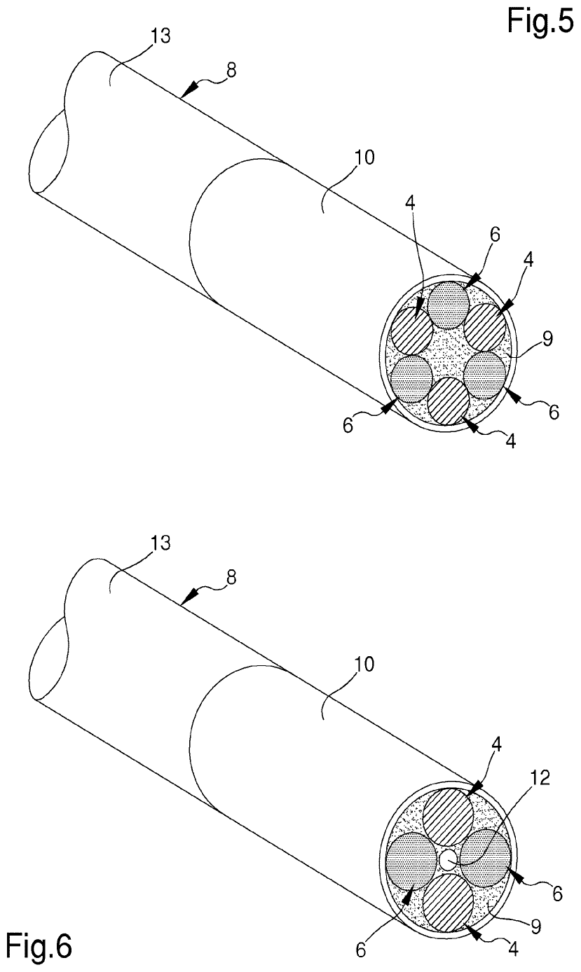

In accordance with alternative embodiments shown in FIGS. 5 and 6, the receiving means 2b comprise a plurality of receiving optical fibers 6.

In detail, the fibers 4,6 are arranged alternately along the circumference of the probe 2 and lack a central fiber 4,6 (FIGS. 5 and 6).

The above arrangements of the fibers 4,6 allow for the even emission of the electromagnetic radiation towards the endometrial mucosa and, consequently, equally even transmission of the reflected and/or scattered electromagnetic radiation.

Each of the optical fibers 4,6 comprises a free ending portion 7 intended to be inserted into the uterus and adapted to emit/receive the electromagnetic radiation.

The opposite ending portion is connected, in one case, to the radiation source 5 and, in the other case, to the optical detecting means 3.

The probe 2 comprises an insertion portion 8 at the ending portions 7.

In accordance with a third embodiment shown in FIG. 6, the device 1 also comprises sensor means 12 associated with the insertion portion 8 and configured to detect chemical and physical parameters representative of the uterus.

The sensor means 12 may comprise, e.g., a thermistor for the measurement of the temperature (electronic measurement) or an electrode for the measurement of pH and/or oxygen saturation and/or carbon dioxide saturation (electrochemical measurement).

Conveniently, the insertion portion 8 also comprises adhesion means adapted to hold the ending portions 7 together.

The adhesion means allow promoting the insertion of the insertion portion 8 into the uterus.

The adhesion means comprise at least one biocompatible epoxy resin 9 having a high transmission index.

The adhesion means also comprise at least one coating element 13 adapted to coat the electromagnetic fibers 4,6. The coating element is made of a flexible material, e.g. a plastic material.

In combination with or as an alternative thereof, the adhesion means comprise at least one retaining element 10 adapted to hold the ends of the optical fibers 4,6 together and possibly the sensor means 12.

The retaining element 10 allows providing high robustness to the probe 2 in the insertion portion and improves the resistance thereof to the sterilization treatments.

The retaining element 10 is in turn attached to the optical fibers by means of biocompatible epoxy resin 9.

In accordance with the preferred embodiment, the retaining element 10 consists of a cylindrical body within which the ends of the optical fibers 4,6 and possibly of the sensor means 12 are fitted.

The retaining element 10 is made of a biocompatible material.

Preferably, the retaining element 10 is made of biocompatible metal material. For example, the retaining element 10 is made of stainless steel.

Conveniently, the diameter of the insertion portion 8 is less than 2.0 mm, preferably less than 1.5 mm.

Advantageously, the probe 2 comprises a catheter 11 which is adapted to allow the insertion of the insertion portion 8 into the uterus.

The insertion portion 8 is arranged within the catheter 11 and is movable with respect thereto.

In detail, the insertion portion 8 is inserted into the catheter 11 and slides with respect thereto to position itself at a predefined distance from the endometrial mucosa during use.

As stated above, the device 1 comprises optical detecting means 3 adapted to process the reflected and/or scattered electromagnetic radiation.

The reflected and/or scattered electromagnetic radiation, transmitted by the receiving optical fiber 6, is further transmitted by the detecting optical fiber until it reaches the inlet slit of the optical analyzer in the range of interest, such as a spectrophotometer, where it is split into the wavelengths of interest by a concave diffraction grating. From there, radiation having the same wavelength strikes a specific pixel on the detector array. On each pixel, the electromagnetic radiation is accumulated by means of an integrative circuit and then converted to digital format, thus providing the absorption spectrum.

The absorption values, depending on the absorption intensities and on the shape of the bands at specific wavelengths, are then converted into values of the investigated chemical and physical parameters by means of the appropriate software.

Some of the physical-chemical parameters can also be measured with different technologies, e.g., temperature with an electronic measurement or oxygen or carbon dioxide saturation with an electrochemical measurement. The parameters are then combined together to obtain the best estimate of endouterine parameters.

According to a further aspect, the present invention also relates to a method implemented by computer for the measurement of endouterine parameters.

The method according to the invention comprises the following stages:
acquisition of an electromagnetic spectrum of a uterine tissue by means of a measuring device according to one or more of the embodiments described above;
generation of a numerical value which is representative of at least one chemical and physical parameter of the uterine tissue by means of a supervised predictive machine learning model from the electromagnetic spectrum.

Conveniently, the machine learning model belongs to the class of Kernel methods, preferably it is a support vector machine.

Specifically, the machine learning model uses radial-based kernel functions. Advantageously, the method comprises a training stage of the predictive model comprising a step of supplying a plurality of electromagnetic spectra of corresponding uterine tissues as input to the supervised predictive machine learning model, in combination with corresponding numerical values representative of at least one chemical and physical parameter of uterine tissues. In accordance with a preferred embodiment, the electromagnetic spectrum is an infrared spectrum and the chemical and physical parameter is the tissue oxygenation parameter (SpO₂).

The starting point for the creation of the predictive model is a data set consisting of N spectra (acquired with the measuring device), with which are associated the relevant N tissue oxygenation reference values acquired with a certified oximeter instrument.

In consideration of the non-linearity between the acquired spectral data item and the reference values, it was decided to use a mathematical model of the nonlinear type called Support Vector Machine (SVM), which is an algorithm belonging to the category of supervised machine learning models called Machine Learning. This type of model is a branch of Artificial Intelligence. More specifically, SVM models fall a class of Machine Learning algorithms referred to as kernel methods, i.e., methods that, through the use of specific mathematical functions, called kernel functions, map data into a different and often higher dimensional space, with the goal of g both classification and regression problems due to the nonlinearity of the input data item.

The kernel functions most commonly used in the context of the SVM models are the linear kernel, the polynomial kernel and the radial-based kernel (called RBF or Gaussian), and their use depends on the type of data item then used for learning the model itself (training set).

In the present case, the best performance in determining the tissue oxygenation values was obtained by using an RBF kernel.

Advantageously, in order to achieve high accuracy of the predictive model, the training stage also comprises a pre-processing step of the electromagnetic spectrum that initially involves a sub-step of calculating the derivative of the electromagnetic spectrum.

The electromagnetic spectrum is in fact represented by a curved line and the derivative allows the slope of the curve to be determined point by point.

Preferably, the derivative function is calculated depending on the values of the spectrum over two wavelength points at a predefined interval, also called a “step”.

Specifically, the derivative function is calculated depending on the values of the spectrum over two wavelength points at an interval of 2, i.e., it is a step 2 derivative function. In actual facts, for each i-th wavelength, the difference between the value of the spectrum at wavelength i and the value of the spectrum at wavelength i+2 is calculated.

The pre-processing step of the electromagnetic spectrum also comprises a sub-step of calculating the mean and standard deviation of the electromagnetic spectra for each wavelength.

Once the derivative is applied, the mean and standard deviation of the spectra making up the dataset at the wavelength level is calculated. Then these values are used to normalize the spectra by subtracting the mean and dividing by the standard deviation.

Pre-processing is also carried out prior to the generation stage, for each electromagnetic spectrum to be analyzed.

To calculate the numerical value of the chemical and physical parameter, the spectrum acquired by the measuring device, after being pre-processed, is mapped into the new space by using the Kernel function and finally processed by applying the prediction coefficients of the predictive model.

It has in practice been ascertained that the described invention achieves the intended objects, and in particular, the fact is emphasized that the device according to the invention for the measurement of endouterine parameters enables effective and accurate detection of the chemical and physical parameters within a uterus. In fact, the acquisition of optical information allows for highly accurate detection of the condition of the endometrial mucosa.

In addition, the present device enables practical, in vivo detection of the endouterine chemical and physical parameters. Indeed, the present device enables the detection of a plurality of parameters by means of a single detection. Finally, the use of fiber optics allows for a significant reduction in the size of the insertion portion, thus allowing for rapid measurement that is well tolerated by the patient.

1) Device (1) for the measurement of endouterine parameters, characterized by the fact that it comprises:
at least one probe (2) insertable at least partly within a patient's uterus and comprising emitting means (2a) adapted to emit electromagnetic radiation to said uterus and receiving means (2b) adapted to receive reflected and/or scattered electromagnetic radiation from said uterus; and
optical detecting means (3) connected to said probe (2) and adapted to process said reflected and/or scattered electromagnetic radiation and to generate an electromagnetic spectrum representative of chemical and physical parameters of said uterus.

2) Device (1) according to claim 1, characterized by the fact that said optical detecting means (3) comprise infrared detection means configured to generate an infrared absorption spectrum.

3) Device (1) according to claim 1, characterized by the fact that said emitting means (2a) comprise at least one emitting optical fiber (4) connected to a radiation source (5) and adapted to emit said electromagnetic radiation and by the fact that said receiving means (2b) comprise at least one receiving optical fiber (6) adapted to receive said reflected and/or scattered electromagnetic radiation and to transmit it to said optical detecting means (3).

4) Device (1) according to claim 1, characterized by the fact that each of said optical fibers (4,6) comprises at least one central core configured to transmit electromagnetic radiation having a wavelength of between 300 nm and 2200 nm and an outer casing to said central core.

5) Device (1) according to claim 1, characterized by the fact that said receiving optical fiber (6) has a section wherein the total diameter defined by said central core and said casing is of between 100 μm and 1 mm, the diameter of said central core being of between 30 μm and 900 μm.

6) Device (1) according to claim 1, characterized by the fact that said receiving optical fiber (6) has a coating made of aluminum.

7) Device (1) according to claim 1, characterized by the fact that said emitting optical fiber (4) has a section wherein the total diameter defined by said central core and said casing is of between 100 μm and 1 mm, where the diameter of said central core is of between 30 μm and 900 μm.

8) Device (1) according to claim 1, characterized by the fact that said emitting optical fiber (4) has a coating made of polyimide.

9) Device (1) according to claim 1, characterized by the fact that said emitting means (2a) comprise a plurality of said emitting optical fibers (4).

10) Device (1) according to claim 1, characterized by the fact that said emitting optical fibers are arranged around said receiving optical fiber (6).

11) Device (1) according to claim 1, characterized by the fact that each of said optical fibers (4,6) comprises an ending portion (7) intended to be inserted into said uterus and adapted to emit/receive said electromagnetic radiation, said probe (2) comprising an insertion portion (8) at said ending portions (7).

12) Device (1) according to claim 1, characterized by the fact that said insertion portion (8) comprises adhesion means adapted to hold said ending portions (7) together, said adhesion means comprising at least one biocompatible epoxy resin (9) having a high transmission index.

13) Device (1) according to claim 1, characterized by the fact that said adhesion means comprise at least one coating element (13) adapted to coat said electromagnetic fibers (4,6) and made of a flexible material.

14) Device (1) according to claim 1, characterized by the fact that said adhesion means comprise at least one retaining element (10) adapted to hold the ends of said optical fibers (4,6) together.

15) Device (1) according to claim 1, characterized by the fact that said retaining element (10) consists of a cylindrical body within which said ends are fitted.

16) Device (1) according to claim 1, characterized by the fact that said retaining element (10) is made of biocompatible metal material.

17) Device (1) according to claim 1, characterized by the fact that it comprises sensor means (12) associated with said insertion portion (8) and configured to detect chemical and physical parameters representative of said uterus.

18) Device (1) according to claim 1, characterized by the fact that said probe (2) comprises a catheter (11) adapted to allow the insertion of said insertion portion (8) into said uterus, said insertion portion (8) being arranged within said catheter (11) and being movable with respect thereto.

19) Method implemented by computer for the measurement of endouterine parameters, characterized by the fact that it comprises the following stages:
acquisition of an electromagnetic spectrum of a uterine tissue by means of a measuring device according to claim 1;
generation of a numerical value which is representative of at least one chemical and physical parameter of said uterine tissue by means of a supervised predictive machine learning model from said electromagnetic spectrum.

20) Method according to claim 19, characterized by the fact that, prior to said generation stage, it comprises a pre-processing stage of said electromagnetic spectrum comprising the following steps:
calculating the derivative of the electromagnetic spectrum;
calculating the mean and standard deviation of the electromagnetic spectra for each wavelength.

21) (canceled)

22) (canceled)

23) (canceled)
